(19) United States
(12) Patent Application Publication
(10) Pub. No.: US 2006/0193812 A1
(43) Pub. Date: Aug. 31, 2006

(54) Controlled delivery system for fragrance comprising a (METH)acrylate/hydroxy (METH) acrylate copolymer

(76) Inventors: Gunter Holzner, Grand-Lancy (CH); Nathalie Pinel, Neydens (FR)

Correspondence Address: WINSTON & STRAWN LLP, 1700 K STREET, N.W., WASHINGTON, DC, 20006, US

(21) Appl. No.: 11/386,966

(22) Filed: Mar. 21, 2006

Related U.S. Application Data
(63) Continuation of application No. PCT/IB04/03160, filed on Sep. 27, 2004.

(30) Foreign Application Priority Data
Oct. 2, 2003 (WO) PCT/IB03/04378

(51) Int. Cl. A61K 8/81

(52) U.S. Cl. 424/70.16; 512/1

(57) ABSTRACT

A perfuming composition for application to the skin by means of a roll-on, pump-spray, squeeze bottle or aerosol spray type device. The composition includes a perfuming base and, optionally, a deodorant or antiperspirant base, with at least one of the bases being combined with a film-forming substrate and an emulsifier so as to form an emulsion. The emulsion, once dried on the skin, provides for in-situ encapsulation of the perfuming base so as to prevent release of the latter until a surge of skin perspiration or contact with a moisture source takes place, and for in-situ re-encapsulation of the perfuming base upon the subsequent drying of the skin. The film-forming substrate is preferably an acrylates/hydroxyacrylates copolymer that is used with other components or that is used alone.

### CROSS-REFERENCE TO RELATED APPLICATIONS

This application is a continuation of International application PCT/IB2004/003160 filed Sep. 27, 2004, the entire content of which is expressly incorporated herein by reference.

### TECHNICAL FIELD

The present invention relates to a controlled delivery system for fragrance, particularly adapted to be used in deodorant and antiperspirant end products that one desires to perfume. The delivery system of the invention is an aqueous based perfuming composition comprising a film forming material and intended for use in perfumed articles and devices. The composition makes it possible to suppress body malodors 01through topical application thereof on the human body skin and provides advantageous odor effects by releasing the constituents of the perfume at the appropriate moment through the action of a source of moisture, in particular sweat. It is suitable for the manufacture of articles for personal care such as deodorants and antiperspirants in the form of a roll-on, pump-spray, squeeze bottle or aerosol spray.

### BACKGROUND

U.S. Pat. No. 4,803,195, belonging to Firmenich S A, describes a perfuming composition having deodorant or antiperspirant activity and the main advantage of which resides in the fact that it makes it possible to control the activation and diffusion of the perfume in time. The contents of the cited patent are hereby included by reference. This patent indicates how, thanks to the particular choice of the ingredients present in the composition, namely of the film-forming substrates and of the emulsifiers, it is possible to achieve reversible “re-encapsulation” of the active deodorant or antiperspirant perfume ingredients, such that successive activation of the perfume can take place on the skin, without any need for further applications by the user. Thus, re-encapsulation takes place in situ during the drying of the skin following a perspiration period.

European patent application EP 384 034-A2 describes a similar composition in which the use of polyvinyl pyrrolidone as the film-forming component made it possible to obtain perfectly transparent alcoholic compositions, particularly useful in applications such as sticks and roll-ons, for which it was also desired to fulfill esthetical requirements. The contents of the cited application are also hereby included by reference.

U.S. Pat. No. 4,818,522 describes a malodor-reducing composition in which a water-immiscible adjuvant, which can be a fragrance, is encapsulated in an antiperspirant active material, said material providing slow release of the encapsulated adjuvant when in contact with moisture. This composition also provides protection of the encapsulated adjuvant during storage.

U.S. Pat. No. 5,135,747 discloses a malodor-reducing composition for body-care, which includes an unscented malodor counteractant mixture encapsulated within a semi-permeable wall, a non-encapsulated fragrant perfume mixture and a cosmetically acceptable vehicle. The encapsulated unscented composition slowly releases its malodor counteractant agent over a period of time, while the fragrant perfume provides a sensory impression.

U.S. Pat. No. 5,711,941 describes underarm hygiene products containing perfumes encapsulated in a film-forming encapsulation material which is capable of re-encapsulating the perfume in an allegedly more efficient manner than the encapsulation materials disclosed in U.S. Pat. No. 4,803,195. It resorts to the use of a film-forming substrate capable of emulsifying the perfume, such that it doesn't require an additional emulsifier.

Other examples of compositions containing film-forming ingredients capable of delivering antibacterial perfumes can be found in U.S. Pat. No. 5,420,104.

In spite of the abundant prior art relating to such types of perfume delivery systems, there is a need to improve aqueous based perfume emulsions of this type, in particular because of the difficulty to stabilize such emulsions, in particular deodorant and antiperspirant media, and so as to optimize the perception of the fragrance by the user once it has been applied to the skin.

We have now established that the use, in the context of the prior described perfume delivery systems, of particular film-forming ingredients provided surprising results as far as repeated perception of the fragrance is concerned. The present invention thus provides an aqueous based carrier system for perfumes, capable of delivering and re-encapsulating the fragrance applied on the skin in a similar manner as that provided by the compositions prior described in U.S. Pat. No. 4,803,195 or EP-A2-384034, so as to protect the active ingredients of the perfume from the action of particularly aggressive agents as in the case of antiperspirants, whilst ensuring efficient release of the perfume over time.

### SUMMARY OF THE INVENTION

The instant invention relates to the perfumery and cosmetic industries. In particular, it provides an aqueous based fragrance delivery system intended for use in perfumed deodorants and antiperspirants. The composition of the invention comprises at least one fragrance and a film-forming ingredient consisting of an acrylates/hydroxyacrylates copolymer.

This invention further relates to a method for suppressing body malodors or perfuming the body, which comprises treating the skin of the human body through topical application of a delivery system as mentioned above.

Another object of the invention is to provide a method for improving the sensory impact of a perfumed product, which comprises incorporating therein a fragrance effective amount of a fragrance delivery composition as mentioned above.

The invention further concerns deodorant and antiperspirant articles comprising the fragrance delivery system of the invention.

### BRIEF DESCRIPTION OF THE DRAWING FIGURES

FIGS. 1 and 2 are graphical results of comparative evaluations by a panel of perfuming experts of certain compositions according to the invention and the prior art.

### DETAILED DESCRIPTION OF THE PREFERRED EMBODIMENTS

This invention relates to an improvement in a perfuming composition for application to the skin by means of a roll-on, pump-spray, squeeze bottle or aerosol spray type device, said composition comprising a perfuming base and, optionally, a deodorant or antiperspirant base, at least one of said bases being combined with a film-forming substrate and an emulsifier so as to form an aqueous emulsion, said emulsion, once dried on the skin, providing for in-situ encapsulation of said perfuming base so as to prevent release of the latter until a surge of skin perspiration takes place, and for in-situ re-encapsulation of said perfuming base upon the subsequent drying of the skin, which improvement consists in that said that said film-forming substrate is, or contains, an acrylates/hydroxyacrylates copolymer.

As it has already been mentioned, the composition of the present invention combines the need for protecting the active ingredients of the perfume from the action of its environment, particularly aggressive in the case of antiperspirants, and the wish to prolong the period of diffusion of the latter. This double action is a result of the phenomenon of in-situ encapsulation described above. When applied to the skin, the perfume is first held at the surface of the emulsion by adhesion, by virtue of the binding effect of the film-forming substrate. Subsequent drying of the skin, effected simply by the exposure to air of the skin thus treated and assisted by the body heat, leads to the active perfuming base being held in the form of microscopic droplets covered with a protective water-soluble film layer formed by the film-forming substrate once dried.

In this case, a simple system is involved which does not require the application of special devices for its use. Any conventional system of current use in cosmetics and currently used for the application of deodorants and antiperspirants can be employed, but preferred embodiments include roll-on, pump-spray and aerosol spray type articles. A most convenient embodiment of the invention is in the form of a deodorant or antiperspirant roll-on or stick comprising the perfume emulsion of the invention.

According to an embodiment of the invention's composition, the film-forming substrate consists essentially of an acrylates/hydroxyacrylates copolymer. According to another, preferred embodiment, the film-forming substrate contains, other than the acrylates/hydroxyacrylates copolymer, one or more compounds selected from the group consisting of the quaternary ammonium compounds, the quaternary cationic polymers, the quaternary cationic silicones, the quaternary cationic celluloses, the gums, the hydrophilic colloids and cellulose derivatives.

By an acrylates/hydroxyacrylates copolymer, we mean here a polymer derived from acrylic and/or methacrylic acid, of which the acrylates/hydroxyesters acrylates copolymers such as for example the products sold by the Rohm & Haas Company, Inc. under the trade name Acudyne®, and more particularly Acudyne®180, are examples. These are typically copolymers of one or more monomers consisting of acrylic acid, methacrylic acid, or their simple esters, and one or more monomers of hydroxyacrylate esters.

Another suitable group of such acrylates/hydroxyacrylates copolymers are the copolymers of polyacrylates/C1-2 succinates/hydroxyacrylates commercialized by Rohm & Haas Company, Inc. under the trade name Allianz®, namely Allianz®LT-120 which is a low viscosity aqueous emulsion polymer, made of a random, linear structure copolymer of C1-2 esters of succinic acid, hydroxyalkyl acrylates, and one or more monomers of acrylic acid, methacrylic acid, or one of their simple esters.

These copolymers are compatible with each other and can be used alone or in admixture.

According to the invention, they can be used in the perfumed emulsion in concentrations varying from 0.1 to 20% of the weight of the emulsion, more preferably from 0.1 to 10% by weight, or even from 0.1 to 5% of the total weight of the emulsion. These concentrations can be easily adjusted by the skilled person as a function of the final deodorant or antiperspirant product that it is desired to obtain and the above-mentioned concentration limits are given by way of example only and should not be taken as limiting the range of concentration values possibly useful.

As an emulsifying agent, mono- or diglycerides of fatty acids, esters derived from the combination of fatty acids with sorbitol or a monosaccharide, or their alkoxylated derivatives, or an ester of tartaric, citric, ascorbic or lactic acid, or yet an alkoxylated alcohol, may be used. The prior art mentioned previously, i.e. U.S. Pat. No. 4,803,195, EP 348034 and U.S. Pat. No. 5,420,104, cites many examples of suitable emulsifiers to be used according to the invention and which can be easily used by the skilled person in the art without undue effort. Other typical examples of emulsifiers that are current in the art of deodorant and antiperspirant preparation can be abundantly found in the patent literature, of which the U.S. 2003/0133891 A1 document is a recent and particularly useful example, the contents of which, namely as far as the pertinent emulsifier citations in pages 4 and 5 are concerned, are herein included by reference.

Other suitable emulsifiers can easily be selected amongst the current ingredients cited in the CTFA reference.

The amount of emulsifier in the composition of the invention can vary in a wide range of values, easily selected as a function of the other ingredients present. By way of example, one can cite typical values of 0.1 to 20% by weight of emulsifier, relative to the total weight of the composition.

The composition according to the invention also contains a perfuming base. In the terms of the present invention, “perfuming base” is to be understood as any perfuming substance or mixture of perfuming substances, whether in isolation or in a solution or suspension in their natural diluents, solvents or co-ingredients. This term includes, in particular, organic solutions which are not generally miscible in water and which have a high vapor tension. Such perfuming bases can be formed by compounds belonging to distinct chemical classes and including, for example, esters, ethers, alcohols, aldehydes, ketones, acids, acetals, nitrites, terpenic hydrocarbons, nitrogen- or sulphur-containing heterocyclic compounds and essential oils of natural or synthetic origin. The particular choice of the perfuming base depends on the perfuming effet required, the nature of the product to be perfumed, and of course, on the taste and preference of the perfumer in question.

Typical examples of usable perfuming compounds are given in the literature and, in this context, S. Arctander, Perfume and Flavor Chemicals, Montclair, N.J. (USA) (1969) may be cited.

Many more examples of materials of current use in perfumery can also be found in the patent literature and the nature of these ingredients is not a determining characteristic of the invention. The perfumer will in fact be able to select such ingredients as a function of their odor and chemical properties, in accordance with the general knowledge in the art and the perfuming effect that he wants to achieve, the odor intensity and tenacity desired and the nature of the other ingredients in the composition.

The concentration of perfume in the composition of the invention can typically vary between 0.1 and 5% by weight.

The composition according to the invention may also contain bactericidal agents with a disinfectant or germicidal action, as well as bacteriostatic agents, particularly when it assumes the form of the end deodorant or antiperspirant product.

Although it is difficult to define a precise range of concentration of the constituents of such deodorants and antiperspirants, they preferably contains (by weight):

a. 0.1 to 20% of film-forming substrate

b. 0.1 to 20% of emulsifying agent

c. 0.1 to 5% of perfuming base and, optionally,

d. 0.1 to 50% of active deodorant or antiperspirant base,

the remainder being water and/or ethanol, inert solvents and/or excipients, and optionally, disinfecting, germicidal or bacteriostatic agents, or yet antioxidants, preservatives and other adjuvants of current use in this type of products.

A deodorant base is to be understood as being a substance capable of masking body odor and inhibiting the growth of the bacteria responsible for decomposing sweat. A considerable number of bactericidal and bacteriostatic products are known and used to this end. By way of example, the following may be used:hexachlorophene, dichlorophenol, tribromosalicylanilide (TBS), tetrachloro-salicylanilide (TCSA), trichlorocarbanilide (TCC) and Triclosan (Irgasan® DP 300, Ciba-Geigy). Alternatively, the deodorizing action of the composition may be achieved just with the perfuming base contained therein, without adding the deodorant base above-mentioned. Other suitable examples are the benzalkonium derivatives or the zinc lactate, phenolsulfate and ricinoleate, commonly cited Deodorant Agents listed in International Cosmetic Ingredient Dictionary & Handbook, Chemical Classes Index, 9^{th} Edition (2002) or in the Cosmetic Toiletry & Fragrance Association website (www.ctfa-online.org/pls/ctfa/ingred_db.class) under the same title. The latter can moreover serve as a guide to the ingredients commonly used as deodorant agents and which are suitable for the invention.

As an antiperspirant base, aluminum salts, for example the above-mentioned aluminum chlorohydrate, are preferably used. Different compositions are proposed on the market as products with an antiperspirant base:Chlorhydrol®, Choracel® and Rezal® or Reach® (Reheis Chem. Co., USA) are examples. These are complex aluminium or aluminium and zirconium salts. Other antiperspirant bases are described in the specialized literature (cf. for example, Herbert P. Fiedler, Der Schweiss, Editio Cantor K G, Aulendorf i. Württ., FRG). Locron® L, an aluminum salt commercialized by Hoechst A G, is a convenient example of a suitable antiperspirant agent, or yet under section “Antiperspirant Agents” of the CTFA document previously mentioned.

Other examples of suitable deodorant and antiperspirants agents of current use in this type of consumer products are extensively cited for example in the above-mentioned U.S. patent application no. 2003/0133891 and the person skilled in the art is referred in particular to the pertinent citations in pages 2 and 3 of said document for guidance. The contents of this document pertaining to the typical components of deodorant and antiperspirant consumer products are herein included by reference.

The perfuming composition according to the invention is particularly suitable for the manufacture of articles intended for personal care. These may occur in many different forms. As mentioned above, preferred embodiments are roll-on, pump-spray and aerosol spray type devices.

According to a preferred embodiment of the invention, the composition will contain, as the film-forming substrate, in addition to the acrylates/hydroxyacrylates copolymer, a second film-forming material selected amongst the quaternarium ammonium compounds and their salts or the cationic polymers generally referred to as “quats”, specifically defined under the title Quaternary Ammonium Compounds of the CTFA Ingredients List mentioned above. As this optional second film-forming component, there can be also used any current cationic polymer such as those cited for example on page 6 of U.S. 2003/0133891, namely cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryldimonium Hydroxypropyl Hydrolyzed Collagen (Lamequat® L, Grünau), quaternized wheat polypeptides, polyethyleneimine, cationic silicone polymers such as, for example, Amodimethicone, copolymers of adipic acid and dimethylamino-hydroxypropyl diethylenetriamine (Cartaretine®, Sandoz), copolymers of acrylic acid with dimethyl diallyl ammonium chloride (Merquat® 550, Chemviron), polyaminopolyamides as described, for example, in FR 2 252 840 A and crosslinked water-soluble polymers thereof, cationic chitin derivatives such as, for example, quaternized chitosan, optionally in micro-crystalline distribution, condensation products of dihaloalkyls, for example dibromobutane, with bis-dialkylamines, for example bis-dimethylamino-1,3-propane, cationic guar gum such as, for example, Jaguar®CBS, Jaguar®C-17, Jaguar®C-16 of Celanese, quaternized ammonium salt polymers such as, for example, Mirapol® A-15, Mirapol® AD-1, Mirapol® AZ-1 of Miranol.

More specific examples of suitable materials to be used as the second film-forming component of the film-forming substrate include the materials belonging to the Quartenium group, in particular the compound commercialized under the Abilquat® 3471 trade name (origin: Degussa, Care Specialities), the materials commercialized by BASF under the trade name Luviquat® or yet the Gafquat® (origin: ISP Corp., USA), Salcare® (origin: Ciba Speciality Chemicals Corp.) and Ultrasil® (origin: Noveon Inc.) type commercial products.

Other suitable materials for the second film-forming material are selected amongst the group defined under the title Gums, Hydrophilic Colloids and Derivatives (including salts) of the CTFA Ingredients List mentioned above, in particular Hydroxypropylcellulose (Klucel®, Hercules Incorporated).

The acrylates/hydroxyacrylates copolymer mentioned above are also compatible with pyrrolidone polymers such as PVP and PVP/VA and the invention also relates to the possible use of mixtures of such compatible materials to achieve particular benefits such as transparency effects in ethanol containing compositions, following the teachings of either EP 384034 or U.S. Pat. No. 5,420,104 (examples 2 and 5 in particular).

The composition according to the invention is obtained by mixing its ingredients by means of conventional apparatuses. The technique of mixing is known per se and any detailed explanation is superfluous here. The method depends essentially on the final article to be manufactured. Specific examples are presented further on and further details can be found in U.S. Pat. No. 4,803,195 or the EP 384034 publication previously cited, the teachings of which are here included by reference.

The invention is illustrated by the following examples, in which the temperatures are indicated in degrees centigrade and the abbreviations have the meanings commonly used in the art.

### EXAMPLES

### Example 1

Antiperspirant Compositions for Roll-on

Antiperspirant compositions to be incorporated in ball-top roll-on-type dispenser containers were prepared using the following ingredients (percentage by weight):

| | | |
|---|---|---|
| A. | Emulgade ® 1000 NI ¹⁾ | 3.00 |
| B. | Water | 74.65 |
| | Locron ®L ²⁾ | 20.00 |
| C. | Acudyne ®180 ³⁾ | 1.00 |
| | Gafquat ®HS 100 ⁴⁾ | 0.35 |
| D. | Perfume | 1.00 |

¹⁾ cetearyl alcohol and ceteareth-20; auto-emulsifying wax from Henkel AG
²⁾ aluminum hydroxychloride; Clariant
³⁾ acrylates/hydroxyesters acrylates copolymer; Rohm & Haas (ISP Products, Inc.?)
⁴⁾ polyquartenium-28; International Speciality Products Corp. (ISP), USA

Part A was heated to 70 C. The part B and part C ingredients were combined separately and then admixed to form a mixture which was then poured into part A under vigorous stirring with a mixer-homogeniser. Stirring was maintained whilst the temperature was allowed to fall down to 40 C. Part D was then added to the mixture and the latter poured into roll-on containers to cool down to ambient (typically 25 C) temperature.

### Example 2

Antiperspirant Compositions for Roll-on

Antiperspirant compositions to be incorporated in ball-top roll-on-type dispenser containers were prepared using the following ingredients (percentage by weight):

| | | |
|---|---|---|
| A. | Steareth-2 ¹⁾ | 2.00 |
| | Steareth-21 ^{1′)} | 1.50 |
| B. | Water | 53.65 |
| | Locron ®L ²⁾ | 40.00 |
| C. | Acudyne ®180 ³⁾ | 1.00 |
| | Luviquat ®Mono CP ⁴⁾ | 0.85 |
| D. | Perfume | 1.00 |

¹⁾ Brij 72 (Unichema Americas)
^{1′)} Brij 721 (Unichema Americas)
²⁾ aluminum hydroxychloride; Clariant
³⁾ acrylates/hydroxyesters acrylates copolymer; Rohm & Haas (ISP Products, Inc.?)
⁴⁾ hydroxyethyl cetyl dimonium phosphate; BASF AG

Part A was heated to 70 C. The part B and part C ingredients were combined separately and then admixed to form a mixture which was then poured into part A under vigourous stirring with a mixer-homogeniser. Stirring was maintained whilst the temperature was allowed to fall down to 40 C. Part D was then added to the mixture and the latter poured into roll-on containers to cool down to ambient (typically 25 C) temperature.

### Example 3

Antiperspirant Compositions for Roll-on

Antiperspirant compositions to be incorporated in ball-top roll-on-type dispenser containers were prepared using the following ingredients (percentage by weight):

| | | |
|---|---|---|
| A. | Emulgade ® 1000 NI ¹⁾ | 3.00 |
| B. | Water | 73.15 |
| | Locron ®L ²⁾ | 20.00 |
| | Klucel ® EF ⁵⁾ | 1.00 |
| | Klucel ® MF ⁶⁾ | 0.50 |
| C. | Acudyne ®180 ³⁾ | 1.00 |
| | Gafquat ®HS 100 ⁴⁾ | 0.35 |
| D. | Perfume | 1.00 |

¹⁾ cetearyl alcohol and ceteareth-20; auto-emulsifying wax from Henkel AG
²⁾ aluminum hydroxychloride; Clariant
³⁾ acrylates/hydroxyesters acrylates copolymer; Rohm & Haas (ISP Products, Inc.?)
⁴⁾ polyquartenium-28; International Speciality Products Corp. (ISP), USA
⁵⁾ and ⁶⁾ hydroxypropylcellulose, Hercules Incorporated

Part A was heated to 70 C. The part B and part C ingredients were combined separately and then admixed to form a mixture which was then poured into part A under vigorous stirring with a mixer-homogeniser. Stirring was maintained whilst the temperature was allowed to fall down to 40 C. Part D was then added to the mixture and the latter poured into roll-on containers to cool down to ambient (typically 25 C) temperature.

### Example 4

A composition prepared according to any of Examples 1 to 3 was applied in the axillary region of 10 male subjects aged between 21 and 36 years. The initial odor released by the skin thus treated was slight, and in certain cases nil, the sprayed solution drying almost instantly. After about two hours, the subjects were involved in intense physical activity, such as represented by 20 minutes of basketball playing. An olfactory assessment carried out at this point showed that the release of perfume was intense. During the ensuing rest period (5 minutes), the drying of the sweat in the air resulted in a considerable decrease in the diffusion of the perfume. When the game resumed, perspiration caused a further release of perfume, which diminished or disappeared after a few minutes drying in the air, as was noted by an evaluation panel at the end of the game.

### Example 5

A composition prepared according to Example 1 (composition B) was compared to a conventional composition (composition A) comprising the same ingredients but wherein the Acudyne® 180 and Gafquat® HS 100 had been entirely replaced by water. The perfume used was the same in both cases, present at a concentration of 1.3% by weight. The two compositions were spread on large glass slides (0.02 g of roll-on antiperspirant) for thermal treatment and evaluation, on a blind test, by a panel of 30 untrained individuals, male and female. The glass slides were left on a thermostatically regulated hot plate at 32 C for 6 h and then evaluated.

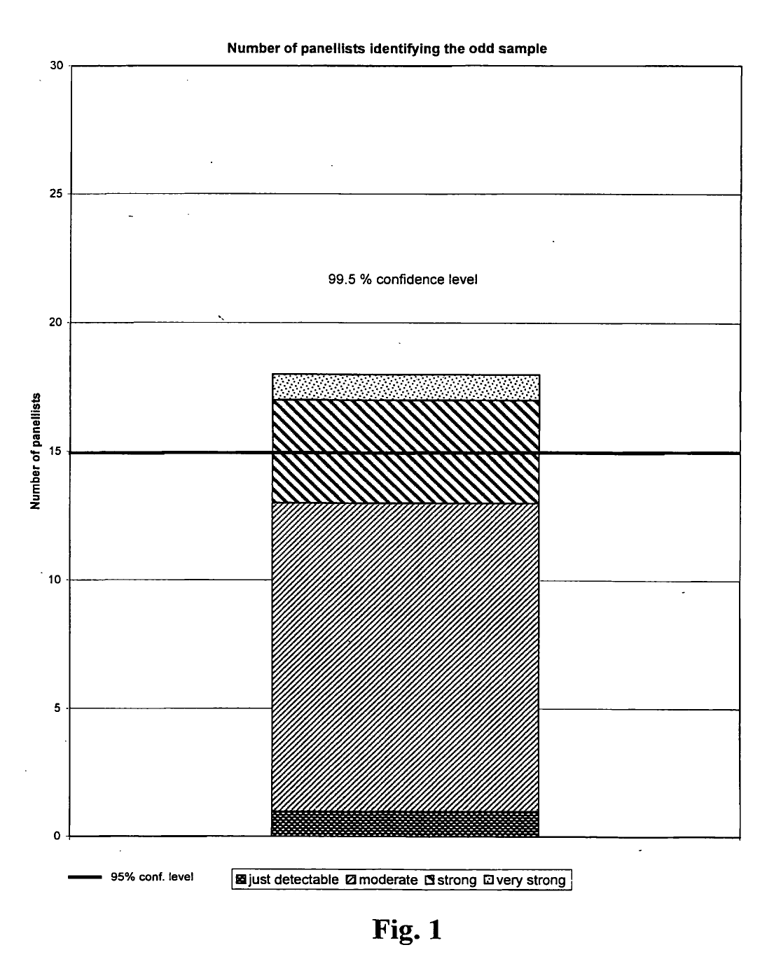

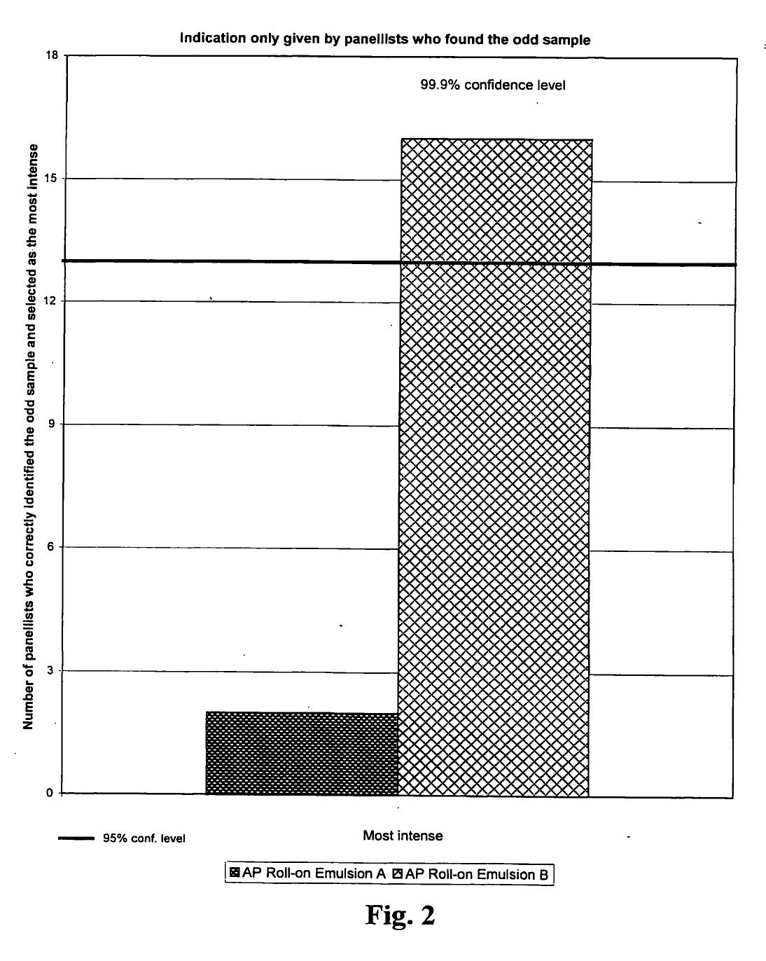

Just before evaluation, a small amount of water (0.05 g) containing 5 g/l of NaCl and 0.5 g/l of urea, was sprayed on the slides from a distance of 5 cm. The compositions were evaluated by the panel via a triangular test, i.e. in which two samples were identical and one different. The panelists were asked to identify the sample which was different and to indicate the degree of difference on a scale of 1 to 4. FIGS. 1 and 2 show the results of this evaluation after 6 h of thermal treatment, as described above.

These results clearly show that, 6 h after application, the panelists are clearly able to identify, in a statistically significant (99.5% confidence level) manner, the substance which is different (FIG. 1). Moreover, amongst the panelists who correctly identified the composition which was different, composition B is perceived with statistical significance (99.9% level), as being stronger than the conventional sample A (FIG. 2).

### Example 6

Similar tests to those described in Example 5 were carried out with a variety of polymers. The panelists were asked to evaluate the quality (A—excellent; B—good, but slightly altered) and strength (on a scale of 1 (weak) to 10 (very strong)) of the odor emanating from glass slides treated for 24 h at 37 C and sprayed as described above. In every case, the compositions according to the invention, comprising Acudyne® 180, or a mixture thereof with the second film-forming component indicated, were compared on a blind triangular test. The perfume used was the same in all the tests and it was present in the compositions at 1.3% by weight.

The following table summarizes the results of the evaluations.

TABLE 1

| Compounds | % by weight | 37° C./24 h |
|---|---|---|
| Luviquat ® | 0.26 | A2 |
| Luviquat ® + Acudyne 180 | 0.26-1.00 | A7 |
| Gafquat ® HS 100 | 0.26 | B3 |
| Gafquat ® HS 100 + Acudyne 180 | 0.35-1.00 | A6 |
| Salcare ® SC 60 | 0.26 | A1 |
| Salcare ® SC 60 + Acudyne 180 | 0.26-1.00 | A6 |
| Abilquat ® 3474 | 0.26 | A5 |
| Abilquat ® 3474 + Acudyne 180 | 0.26-1.00 | A8 |
| Acudyne ® 180 | 0.35 | A5 |

The results shown in this table clearly provide evidence that, for similar concentrations, the strength of perfume perceived after 24 h of application of the composition, is consistently higher whenever the film-forming substrate comprises Acudyne®180.

What is claimed is:

1. A perfuming composition for application to the skin by means of a roll-on, pump-spray, squeeze bottle or aerosol spray type device comprising a perfuming base and, optionally, a deodorant or antiperspirant base, at least one of the bases being combined with a film-forming substrate and an emulsifier so as to form an emulsion, said emulsion, once dried on the skin, providing for in-situ encapsulation of the perfuming base so as to prevent release of the latter until a surge of skin perspiration or contact with a moisture source takes place, and for in-situ re-encapsulation of the perfuming base upon the subsequent drying of the skin, the improvement which consists in that the film-forming substrate comprises an acrylates/hydroxyacrylates copolymer.

2. A perfuming composition according to claim 1, wherein the film-forming substrate consists of the acrylates/hydroxyacrylates copolymer.

3. A perfuming composition according to claim 1, wherein the film-forming substrate contains, other than the acrylates/hydroxyacrylates copolymer, one or more compounds selected from the group consisting of the quaternary ammonium compounds, the quaternary cationic polymers, the quaternary cationic silicones, the quaternary cationic celluloses, the gums, the hydrophilic colloids and cellulose derivatives.

4. A perfuming composition according to claim 1, wherein the acrylates/hydroxyacrylates copolymer is an acrylates/hydroxyesters acrylates copolymer.

5. A perfuming composition according to claim 1, wherein the emulsifying agent is selected from the group consisting of mono- or diglycerides of fatty acids, esters derived from the combination of fatty acids with sorbitol or with a saccharide, or their alkoxylated derivatives, esters of tartaric, citric, ascorbic or lactic acid, and alkoxyalkylated alcohols.

6. A perfuming composition according to claim 1, wherein the emulsion consists of
a. 0.1 to 20% of film-forming substrate
b. 0.1 to 20% of emulsifying agent
c. 0.1 to 5% of perfuming base and, optionally,
d. 0.1 to 50% of active deodorant or antiperspirant base,
the remainder being water, optionally containing one or more of ethanol, inert solvents, excipients, disinfecting, germicidal or bacteriostatic agents.

7. A composition according to claim 6, wherein the emulsion consists of
a. 0.1 to 20% of film-forming substrate
b. 0.1 to 20% of emulsifying agent
c. 0.1 to 5% of perfuming base,
the remainder being water.

8. A perfuming composition according to claim 1, wherein the antiperspirant base consists of an aluminium or zirconium salt.

9. A deodorant or antiperspirant roll-on, pump-spray, squeeze bottle or aerosol spray type device, characterized in that it contains a perfuming composition according to claim 1.

10. A method for suppressing body malodors or perfuming the body, which comprises treating the skin of the human body through topical application, by means of a roll-on, pump-spray, squeeze bottle or aerosol spray type device, of a composition comprising a perfuming base and, optionally, a deodorant or antiperspirant base, at least one of the bases being combined with a film-forming substrate and an emulsifier so as to form an emulsion, said emulsion, once dried on the skin, providing for in-situ encapsulation of said perfuming base so as to prevent release of the latter until a surge of skin perspiration or contact with a moisture source takes place, and for in-situ re-encapsulation of the perfuming base upon the subsequent drying of the skin, the improvement which consists of the film-forming substrate comprising or consisting of an acrylates/hydroxyacrylates copolymer.
